(19) United States
(12) Patent Application Publication
(10) Pub. No.: US 2003/0095935 A1
(43) Pub. Date: May 22, 2003

(54) Transfer resistant cosmetic compositions comprising silicone gels

(75) Inventors: Atchara Chaiyawat, Ballston Lake, NY (US); John Alfred Kilgour, Clifton Park, NY (US); Michael Joseph O'Brien, Clifton Park, NY (US); Kuo-Tsai Griffin Lai, Clifton Park, NY (US); Robert Francis Agars, Clifton Park, NY (US); Stephen J. Gregor, Troy, NY (US)

Correspondence Address: Kenneth S. Wheelock, GE Plastics, One Plastics Avenue, Pittsfield, MA, 01201, US

(73) Assignee: General Electric Company

(21) Appl. No.: 09/995,992

(22) Filed: Nov. 16, 2001

(51) Int. Cl. A61K 7/021; A61K 7/025

(52) U.S. Cl. 424/63; 424/64

(57) ABSTRACT

Color cosmetic formulations employing silicone gels exhibit greater transfer resistance to the transfer of color from a first substrate to which the color cosmetic is applied to a second substrate.

### FIELD OF THE INVENTION

The present invention relates to cosmetic compositions comprising silicone elastomers wherein said cosmetic compositions exhibit the desirable property of forming films that are transfer resistant. More particularly, the invention relates to a transfer-resistant personal care and/or make-up composition for the skin of both the face and the human body, for the mucous membranes such as the lips and the inside of the lower eyelids, or alternatively for the superficial body growths such as the eyelashes, the eyebrows, the nails and the hair. This composition may be provided in particular in the form of a product cast as a stick or in a dish such as lipsticks or lip balms, cast foundations, concealers, eyeshadows or blushers, in the form of a paste or a cream which is fluid to a greater or lesser degree such as fluid foundations or lipsticks, eyeliners, compositions for protecting against sunlight or for coloring the skin.

### BACKGROUND OF THE INVENTION

Make-up or care products for the skin or the lips of human beings such as foundations or lipsticks generally contain lipophilic or fatty phases such as waxes and oils, pigments and/or fillers and, optionally, additives such as cosmetic or dermatological active agents. They may also contain so-called “pasty” products, of soft consistency, which make it possible to obtain pastes, colored or otherwise, to be applied with a brush. These compositions, when they are applied to the skin or the lips, have the disadvantage of transferring, that is to say of becoming deposited at least in part, leaving marks, onto certain supports with which they may be brought into contact, and in particular a glass, a cup, a cigarette, clothing or the skin. This results in poor persistence of the applied film, requiring regular renewed application of the foundation or lipstick composition. Moreover, the appearance of these unacceptable marks especially on blouse collars can prevent some women from using this type of make-up.

For several years, cosmeticians have been interested in lipstick compositions and, more recently, in foundation compositions which are “transfer-free”. Thus, “transfer-free” lipstick compositions containing a siloxy silicate resin (with a three-dimensional network), a volatile silicone oil with a cyclic silicone chain and pulverulent fillers have been described. Likewise, “transfer-free” lipstick, eyeliner and foundation compositions containing one or more volatile silicones combined with one or more hydrocarbon waxes have been developed.

Although exhibiting enhanced “transfer-free” properties, these compositions have the disadvantage of leaving on the lips, after evaporation of the silicone oils, a film which becomes uncomfortable over time (feeling, of dryness and tightness), a significant drawback to the retail purchaser of cosmetics. To enhance the comfort of this type of composition, nonvolatile silicone or nonsilicone oils may be added, but this particular solution to the problem results in a loss of some of the “transfer-free” properties.

More recently, water-in-oil emulsion-type mascara compositions have been described that exhibit long retention, resistance to water and which do not leave marks. These compositions contain, inter alia, a water-insoluble polymer generally called a latex, combined with a surfactant of the alkyl or alkoxy dimethicone copolyol type, hydrocarbon oils, pigments and fillers as well as waxes.

The compositions based on silicone oils and silicone resins as well as those based on latex provide matte colored films. However, many purchasers of cosmetic products are looking for glossy products especially for lip coloring. Furthermore, the transfer-free properties of the films deposited are not perfect. In particular, substantial pressure or rubbing leads to a decrease in the color of the deposit and to redeposition on the support brought into contact with these films.

Compositions containing a styrene-ethylene-propylene block polymer combined with waxes, light or volatile oils and pigments exhibit so-called “transfer-free” properties. However, these compositions have the disadvantage of not being comfortable to any great extent, having poor cosmetic properties, and being difficult to formulate. Moreover, the “transfer-free” properties of these compositions are only average.

A variety of silicone compositions may be utilized to impart transfer resistance to cosmetic compositions and/or formulations, e.g. MQ resins, silicone gums, MQ esters and alkyl silicones. While cosmetics utilizing MQ resins possess the property of transfer resistance, formulations containing most MQ resins exhibit an unpleasant pulling sensation and additionally do not stabilize the formulation. Alkyl silicones offer transfer resistance, enhanced sensory properties and a somewhat reduced syneresis. These advantages come at the price of high levels of the alkyl silicone in the cosmetic composition, e.g. 13-30 wt. %, levels that significantly reduce formulation flexibility.

The need, therefore, still exists for a composition that does not exhibit the above disadvantages, and having in particular “transfer-free” properties even during substantial or intensive pressure or rubbing, an appearance which is glossy to a greater or lesser degree, in line with the wishes of the consumer, which does not cause the skin or the lips to which it is applied to dry out over time.

### SUMMARY OF THE INVENTION

The present invention provides for a colored cosmetic composition comprising;

(a) a silicone gel;

(b) a dispersant medium; and

(c) a colored material

whereby said colored cosmetic composition is transfer resistant. More particularly the present invention provides for a colored cosmetic composition wherein the silicone gel is selected from the group of gels consisting of:

(i) a gel formed from a silicone and a hydrosilylation compatible solvent wherein said silicone is prepared by the hydrosilylation of a linear alkenyl polyorganosiloxane and a hydride resin;

(ii) a gel formed as a reaction product of an epoxy functional hydrido-siloxane said reaction product being formed in an epoxy-gel formation compatible solvent;

(iii) a gel formed from a silicone and a hydrosilylation compatible solvent wherein said silicone is prepared by the hydrosilylation of a linear hydrogen polyorganosiloxane and an alkenyl resin;

(iv) a gel formed from a silicone and a hydrosilylation compatible solvent wherein said silicone is prepared by the hydrosilylation of a linear hydrogen polyorganosiloxane and a linear alkenyl polyorganosiloxane;

(v) a gel formed from a silicone and hydrosilylation compatible solvent wherein said silicone is prepared by the hydrosilylation of a hydrogen polyorganosiloxane resin and an alkenyl polyorganosiloxane resin;

(vi) a gel formed from a silicone and a hydrosilylation compatible solvent wherein said silicone is prepared by the hydrosilylation of a linear hydrogen organopoly-siloxane having two or more hydride functionalities per molecule and an α, ω reactive organic molecule possessing two or more reactive functionalities per molecule; and

(vii) a gel formed as a reaction product of a vinyl functional hydrido-siloxane in a hydrosilylation compatible solvent.

The present invention also provides for colored cosmetic compositions comprising colored materials selected from the group consisting of FD&C blue no. 1, FD&C green no. 3, FD&C red no. 4, FD&C red no. 40, FD&C yellow no. 5, F &C yellow no. 6, D&C blue no. 4, D&C brown no. 1, D&C green no. 5, D&C green no. 6, D&C green no. 8, D&C orange no. 4, D&C orange no. 5, D&C orange no. 10, D&C orange no. 11, D&C red no. 6, D&C red no. 7, D&C red no. 17, D&C red no. 21, D&C red no. 22, D&C red no. 27, D&C red no. 28, D&C red no. 30, D&C red no. 31, D&C red no. 33, D&C red no. 34, D&C red no. 36, D&C violet no. 2, D&C yellow no. 7, D&C yellow no. 8, D&C yellow no. 10, D&C yellow no. 11. Ext. D&C violet no. 2, Ext. D&C yellow no. 7, Iron oxide (red, yellow, black), Titanium dioxide, Zinc oxide, Ultramarine, Bismuth oxychloride, Chromium oxide green, Chromium hydroxide green, Ferric ferrocyanide, Manganese violet, Guanine, Acid green no. 1, Pigment yellow no.1, Pigment yellow no. 3, Solvent red no. 3, Solvent red no. 1, Pigment red no. 112, Pigment red no. 5, Acid orange no. 6, Acid red no. 14, Pigment red no. 68, Pigment red no.48, Acid red no. 27 & Al lake, Acid red no.18, Acid black no. 1, Pigment yellow no. 13, Solvent yellow no. 29, Acid red no. 73, Brilliant black no. 1, Acid blue no. 1, Acid blue no. 3, Basic violet no. 14, Basic blue no.26, Acid green no. 50, Acid red no. 52, Acid violet no. 9, Acid red no. 51, Pigment violet no.23, Pigment red no. 83, Acid blue no. 62, Acid blue no. 74, Pigment violet no. 19, Pigment blue no. 15, Direct blue no. 86, Pigment green no. 7, Bentonite, Barium sulfate, Calcium sulfate, Carbon black, Iron oxide (orange), Magnesium carbonate, Lactoflavin, Capsanthin, capsorubin, Beetroot red, Anthocyanins, Aluminum stearate, Zinc stearate, Magnesium stearate, Calcium stearate, Bromothymol blue, Bromocresol green, Acid red, Color Index (CI) 195, CI 18736, CI 18820, CI 18965, CI 20040, CI 21108, CI 24790, CI 27755, CI 40215, CI 40820, CI 40825, CI 40850, CI 42080, CI 42090, CI 42100, CI 42170, CI 42520, CI 42735, CI 45220, CI 45396, CI 45405, CI 50325, CI 50420, CI 60724, CI 61585, CI 69800, CI 69825, CI 71105, CI 73000, CI 73385, CI 73915, CI 74100, CI 75100, CI 75125, CI 75135, CI 75300, CI 77002, CI 77015, CI 77220, CI 77267, CI 77268:1, CI 77346, CI 77480, CI 77745, Beta carotene, Annatto, Caramel, Carmine, Chlorophyllin—copper complex, Henna, Aluminum powder, Bronze or copper powder, Silver, Mica. and Titanated mica.

### DETAILED DESCRIPTION OF THE INVENTION

Applicants have discovered that colored cosmetic compositions may be rendered transfer resistant by the incorporation of a silicone gel into the formulation of the colored cosmetic composition. As used herein transfer resistance is resistance to the transfer of colored material in a color cosmetic from a first substrate to which the colored cosmetic is applied to a second substrate under the application of a transferring force such as pressure.

As used herein the phrase “silicone gel” refers to any silicone containing material that increases its volume upon contact with a low molecular weight solvent that may or may not be volatile wherein the solvent diffuses into the silicone containing material.

As used herein the terms polyorganosiloxane and organopolysiloxane are interchangeable one with the other.

As used herein the term “colored material” refers to physiologically acceptable dyes, pigments or other coloring material known to the cosmetic arts. The term “physiologically acceptable” is an accepted term of art and refers to topical application on humans.

As used herein the phrase “silicone containing material” refers to polymers, copolymers, terpolymers and higher order polymers of silicon containing repeat units, copolymers and higher order interpolymers containing silicon repeat units with organic polymers. As used herein “organic polymers” means organic polymers wherein the repeat units do not contain silicon atoms in the polymeric backbone or chain. Thus for example, some silicones gels useful in the compositions of the present invention are polymeric, cross-linked, networks of organopolysiloxanes or block copolymers of organopolysiloxanes and organic polymers. The cross-linking of either type of network may be achieved by cross-linking units based on siloxanes comprising hydrido-, vinyl-, epoxy-, acrylate-, acetoxy-, or alkoxy-groups and the like and mixtures thereof and when such materials contain organic polymers as a component, organic compounds or oligomers capable of joining polymeric units together, e.g. terminal polyolefins, terminal polyolefinic ethers, acrylates, epoxides and the like and blends thereof. Silicone gels useful in the compositions of the present invention are exemplified in the following U.S. Pat. Nos. 4,987,169; 4,980,167; 5,760,116; 5,811,487 and 5,138,009 hereby and herewith specifically incorporated by reference.

As used herein the phrase “low molecular weight volatile solvent” refers to any solvent compatible with topical application to human beings without adverse effect thereto that has a vapor pressure between the temperatures of 0° C. and 100° C. ranging from about 1 mm Hg to 760 mm Hg.

The silicone gels utilized in the transfer resistant formulations of the present invention may be prepared in a variety of chemically appropriate solvents (hereinafter defined and listed). Once prepared, the silicone gels may be dispersed in a variety of chemically appropriate solvents (hereinafter defined and listed).

While a variety of silicone gels may be prepared by condensation cure mechanisms, e.g. room temperature vulcanizable compositons. the following specific silicone gels are preferred.

Silicone Gel I.

The present invention may utilize a silicone gel composition comprising:

(A) a first silicone formed by the hydrosilylation product of

(1) a linear alkenyl polyorganosiloxane having the formula:

M^{vi}ₐDₓD^{vi}_{y}M₂₋ₐ

where the subscript x is a number greater than 10, the subscript y is a number ranging from zero to about 20, the subscript a is a number ranging from 0 to 2, subject to the limitation that a+y is within the range of from 1 to about 20. with M^{vi} defined as:

R¹R²R³SiO_{1/2}

where R¹ is a monovalent unsaturated hydrocarbon radical having from two to ten carbon atoms, and R² and R³ are each independently one to forty carbon atom monovalent hydrocarbon radicals, with D defined as:

R⁴R⁵SiO_{2/2}

where R⁴ and R⁵ are each independently one to forty carbon atom monovalent hydrocarbon radicals, with D^{vi} defined as:

D^{vi}=R⁶R⁷SiO_{2/2}

where R⁶ is a monovalent unsaturated hydrocarbon radical having from two to ten carbon atoms, and R⁷ is independently a one to forty carbon atom monovalent hydrocarbon radical with M defined as

M=R⁸R⁹R¹⁰SiO_{1/2}

with R⁸, R⁹, and R¹⁰ each independently a one to forty carbon atom monovalent hydrocarbon radical; and

(2) a hydride resin having the formula:

(M^{H}_{w}Q_{z})ⱼ

where Q has the formula SiO_{4/2} and with M^{H} defined as

H_{b}R¹¹_{3-b}SiO_{1/2}

where R¹¹ is a one to forty carbon atom monovalent hydrocarbon radical where the subscript b is a number ranging from 1 to 3, with the subscripts w and z having a ratio of 0.5 to 4.0 respectively and the subscript j ranges from about 2.0 to about 100; wherein said hydrosilylation is conducted in the presence of

(3) a hydrosilylation compatible solvent preferably a silicone having a viscosity below about 1,000 centistokes at 25° C. or a hydrosilylation compatible lipohilic phase (hereinafter also referred to as a hydrosilylation compatible solvent), thereby forming a gel; and

(B) a lipophilic phase or a silicone having a viscosity below about 1,000 centistokes at 25° C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25° C.

The silicone having a viscosity below about 1,000 centistokes at 25° C. is preferably selected from the group consisting of cyclic silicones having the formula:

D_{f}

where the subscript f is an integer ranging from about three to about 6 with D defined as:

R⁴R⁵SiO_{2/2}

where R⁴ and R⁵ are each independently one to forty carbon atom monovalent hydrocarbon radicals and

linear silicones having the formula:

M′D′ᵢM′

where D′ is defined as:

R⁴R⁵SiO_{2/2}

where R⁴ and R⁵ are each independently one to forty carbon atom monovalent hydrocarbon radicals and M′ has the formula:

R¹²R¹³R¹⁴SiO_{1/2}

where R¹², R¹³ and R¹⁴ are each independently one to forty carbon atom monovalent hydrocarbon radicals.

Silicone Gel II.

Other gels suitable for the compositions of the present invention comprise the reaction products of an epoxy functional hydrido-siloxane molecule having the following formula:

M_{α}M^{H}_{β}M^{E}_{χ}D_{δ}D^{H}_{ε}D^{E}_{φ}T_{γ}T^{H}_{η}T^{E}_{ι}Q_{κ}

where

M=R^{1′}R^{2′}R^{3′}SiO_{1/2};

M^{H}=R^{4′}R^{5′}H SiO_{1/2};

M^{E}=R^{6′}R^{7′}R^{E}SiO_{1/2};

D=R^{8′}R^{9′}SiO_{2/2};

D^{H}=R^{10′}HSiO_{2/2};

D^{E}=R^{11′}R^{E}SiO_{2/2};

T=R^{12′}SiO_{3/2};

T^{H}=HSiO_{3/2};

T^{E}=R^{E}SiO_{3/2}; and

Q=SiO_{4/2};

where R^{1′}, R^{2′}, R^{3′}, R^{8′}, and R^{12′} are independently monovalent hydrocarbon radicals having from one to sixty carbon atoms; R^{4′}, R^{5′} and R^{10′} are independently monovalent hydrocarbon radicals having from one to sixty carbon atoms or hydrogen; R^{6′}, R^{7′}, R^{11′} are independently monovalent hydrocarbon radicals having from one to sixty carbon atoms or R^{E}; each R^{E} is independently a monovalent hydrocarbon radical containing one or more oxirane moieties having from one to sixty carbon atoms; the stoichiometric subscripts α, β, χ, δ, ε, φ, γ, η, ι, and κ are either zero or positive subject to the following limitations: α+β+χ>1; β+εη>1; χ+φ+ι>1; β+ε+η>χ+φ+ι; and when δ+ε+φ+γ+η+ι+κ=0, α+β+χ=2.

The reaction product of an epoxy functional hydrido siloxane molecule is preferably prepared in an epoxy gel formation medium selected from a lipophilic phase or a silicone fluid selected from the group consisting of cyclic silicones having the formula:

D_{f}

where the subscript f is an integer ranging from about three to about 6 with D defined as:

R⁴R⁵SiO_{2/2}

where R⁴ and R⁵ are each independently one to forty carbon atom monovalent hydrocarbon radicals and

linear silicones having the formula:

M′D′ᵢM′

where D′ is defined as:

R⁴R⁵SiO_{2/2}

where R⁴ and R⁵ are each independently one to forty carbon atom monovalent hydrocarbon radicals and M′ has the formula:

R¹²R¹³R¹⁴SiO_{1/2}

where R¹², R¹³ and R¹⁴ are each independently one to forty carbon atom monovalent hydrocarbon radicals.

Once prepared, the type II silicone gels that may be utilized in the composition of the present invention may be slurried and mixed in a dispersant medium selected from a lipophilic phase or a silicone selected from the group consisting of cyclic silicones having the formula

D_{f}

where the subscript f is an integer ranging from about three to about 6 with D defined as

R⁴R⁵SiO_{2/2}

where R⁴ and R⁵ are each independently one to forty carbon atom monovalent hydrocarbon radicals and

linear silicones having the formula

M′D′ᵢM′

where D′ is defined as

R⁴R⁵SiO_{2/2}

where R⁴ and R⁵ are each independently one to forty carbon atom monovalent hydrocarbon radicals and M′ has the formula

R¹²R¹³R¹⁴SiO_{1/2}

where R¹², R¹³ and R¹⁴ are each independently one to forty carbon atom monovalent hydrocarbon radicals.

Silicone Gel III.

The present invention may utilize a silicone gel composition comprising:

(A) a silicone formed by the hydrosilylation product of

(1) a linear hydrogen polyorganosiloxane having the formula:

M^{H}ₐDₓD^{H}_{y}M₂₋ₐ

where the subscript x is a number greater than 10, the subscript y is a number ranging from zero to about 20, the subscript a is a number ranging from 0 to 2, subject to the limitation that a+y is within the range of from 1 to about 20, with M^{H} defined as:

R¹R²R³SiO_{1/2}

where R¹ is hydrogen, R² and R³ are each independently one to forty carbon atom monovalent hydrocarbon radicals, with D defined as:

R⁴R⁵SiO_{2/2}

where R⁴ and R⁵ are each independently one to forty carbon atom monovalent hydrocarbon radicals, with D^{H} defined as:

D^{H}=R⁶R⁷SiO_{2/2}

where R⁶ is hydrogen and R⁷ is independently a one to forty carbon atom monovalent hydrocarbon radical with M defined as

M=R⁸R⁹R¹⁰SiO_{1/2}

with R⁸, R⁹, and R¹⁰ each independently a one to forty carbon atom monovalent hydrocarbon radical; and

(2) an alkenyl resin having the formula:

(M^{vi}_{w}Q_{z})ⱼ

where Q has the formula SiO_{4/2} and with M^{vi} defined as

R¹¹_{b}R¹²_{3-b}SiO_{1/2}

where R¹¹ is a monovalent unsaturated hydrocarbon radical having from two to ten carbon atoms, R¹² is a one to forty carbon atom monovalent hydrocarbon radical where the subscript b is a number ranging from 1 to 3, with the subscripts w and z having a ratio of 0.5 to 4.0 respectively and the subscript j ranges from about 2.0 to about 100; wherein said hydrosilylation is conducted in the presence of

(3) a hydrosilylation compatible solvent preferably a silicone having a viscosity below about 1,000 centistokes at 25° C. or a hydrosilylation compatible lipohilic phase (hereinafter also referred to as hydrosilylation compatible solvent), thereby forming a gel; and

(B) a lipophilic phase or a silicone having a viscosity below about 1,000 centistokes at 25° C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25° C.

Silicone Gel IV

A first silicone being the hydrosilylation reaction product of a linear alkenyl organopolysiloxane (as previously defined) having two or more alkenyl functionalities per molecule as above with a linear hydrogen organopolysiloxane (as previously defined) having two or more hydrogen functionalities per molecule prepared as above in the presence of a hydrosilylation compatible solvent or silicone, D_{f} and/or M′D′ᵢM′ where D_{f} and M′D′ᵢM′ are as previously defined. The gel as prepared may then be slurried with a lipophilic phase or a silicone having a viscosity below about 1,000 centistokes at 25° C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25° C.

Silicone Gel V

A first silicone being the hydrosilylation reaction product of an alkenyl organopolysiloxane resin having two or more alkenyl functionalities per molecule having the formula

M^{vi}_{c}D_{d}D^{vi}ₑT_{g}T^{vi}ₖM_{2-c}M″_{g+k}

with a hydrogen organopolysiloxane resin having two or more hydrogen functionalities per molecule having the formula

M^{H}ₙDₚD^{H}ᵣTₛT^{H}ᵤMᵥM″ₛ₊ᵤ

where all the terms are as previously defined with

T=R¹⁶SiO_{3/2} where R¹⁶ is a one to forty carbon atom monovalent hydrocarbon radicals;

T^{vi}=R¹⁷SiO_{3/2} where R¹⁷ is a monovalent unsaturated hydrocarbon radical having from two to forty carbon atoms;

T^{H}=HSiO_{3/2};

M″ is independently M^{H}, M^{vi} or M and the subscripts c, d, e, g, k, n, p, r, s, u, and v are either zero or positive subject to the limitations that g+k+s+u is ≧1; c+e+k ≧2 and n+r+u≧2; prepared in a hydrosilylation compatible solvent and slurried in a lipophilic phase or a silicone having a viscosity below about 1,000 centistokes at 25° C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25° C.

Silicone Gel VI

A first silicone being the reaction product of a linear hydrido organopolysiloxane having two or more hydride functionalities per molecule with an α, ω reactive organic molecule possessing two or more reactive functionalities per molecule in the presence of a lipophilic phase or second silicone, D_{f} and/or M′D′ᵢM′ where D_{f} and M′D′ᵢM′ are as previously defined. The reactive functionalities of the α, ω reactive organic molecule possessing two or more functionalities per molecule are selected from the group of organic functional groups consisting of olefins, acetylenes, vinylethers, acrylates or acrylate esters (eg CH2=CHCOOROCOCH=CH2), and alcohols and the like. Thus the α, ω reactive organic molecule possessing two or more functionalities per molecule subtends a large group of organic molecules that includes α, ω-di-olefins, α, ω-olefins possessing a polyolefinic functionality, α, ω-di-acetylenes, α, ω-di-acetylenes possessing a polyacetylenic functionality, including side chain substituted variations where the side chains possess reactive functionality as herein defined. This gel is prepared in a hydrosilylation compatible solvent and slurried in a lipophilic phase or a silicone having a viscosity below about 1,000 centistokes at 25° C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25 ° C.

Silicone Gel VII

Other gels suitable for the compositions of the present invention comprise the reaction products of a vinyl functional hydrido-siloxane molecule having the following formula:

M_{a′}M^{vi}_{b′}M^{H}_{c′}D_{d′}D^{vi}_{e′}D^{H}_{f′}T_{g′}T^{vi}_{h′}T^{H}_{i′}Q_{j′}

where all the terms are as previously defined and the subscripts a′, b′, c′, d′, e′, f′, g′, h′, i′ and j′ are either 0 or a positive integer for well defined molecular species subject to the limitation b′+e′+h′ is greater than or equal to one and further subject to the limitation that c′+f′+i′ is greater than or equal to one. Mixtures of compounds where individual molecular species possess the formula, M_{a′}M^{vi}_{b′}M^{H}_{c′}D_{d′}D^{vi}_{e′}D^{H}_{f′}T_{g′}T^{vi}_{h′}T^{H}_{i′}Q_{j′}, will analyze for non-integral values of the subscripts because of the fact that it is a mixture and not a pure compound. Thus for mixtures of compounds possessing the formula. M_{a′}M^{vi}_{b′}M^{H}_{c′}D_{d′}D^{vi}_{e′}D^{H}_{f′}T_{g′}T^{vi}_{h′}T^{H}_{i′}Q_{j′}, the subscripts a′, b′, c′, d′, e′, f′, g′, h′, i′ and j′ will be zero or positive. Compounds possessing the formula M_{a′}M^{vi}_{b′}M^{H}_{c′}D_{d′}D^{vi}_{e′}D^{H}_{f′}T_{g′}T^{vi}_{h′}T^{H}_{i′}Q_{j′} may be prepared by the procedures and methods disclosed in U.S. Pat. Nos. 5,698,654; 5,753,751; and 5,965,683 herewith specifically incorporated by reference. These materials may be reacted with the silicone precursors to the class II silicone gels previously defined herein or they may self-reacted in the presence of a noble metal hydrosilylation catalyst as is known in the art. These materials are prepared in a hydrosilyation compatible solvent and slurried in a lipophilic phase or a silicone having a viscosity below about 1,000 centistokes at 25° C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25° C.

Many types of noble metal catalysts for hydrosilylation (or SiH olefin addition reaction) are known and such noble metal catalysts may be used for the preparative reactions involved in making the compositions of the present invention. The most preferred noble metals are those of the platinum group metals, specifically rhodium and platinum. When optical clarity of the resulting addition product is required the preferred catalysts are those catalysts that are compounds that are soluble in the reaction mixture. One such platinum compound can be selected from those having the formula (PtCl₂Olefin) and H(PtCl₃Olefin) as described in U.S. Pat. No. 3,159,601, hereby incorporated by reference. The olefin shown in the previous two catalyst compound formulas can be almost any type of olefin but is preferably an alkenylene having from 2 to 8 carbon atoms, a cycloalkenylene have from 5 to 7 carbon atoms or styrene. Specific olefins utilizable in the above formulas are ethylene, propylene, the various isomers of butylene, octylene, cyclopentene, cyclohexene, cycloheptene, and the like.

A further platinum containing material usable in the compositions of the present invention is the cyclopropane complex of platinum chloride described in U.S. Pat. No. 3,159,662 hereby incorporated by reference.

Further the platinum containing material can be a complex formed from chloroplatinic acid with up to 2 moles per gram of platinum of a member selected from the class consisting of alcohols, ethers, aldehydes and mixtures of the above as described in U.S. Pat. No. 3,220,972 hereby incorporated by reference.

The catalysts are described in U.S. Pat. Nos. 3,715,334; 3,775,452; and 3,814,730 to Karstedt. Additional background concerning the art may be found at J. L. Spier, “Homogeneous Catalysis of Hydrosilation by Transition Metals, in Advances in Organometallic Chemistry, volume 17, pages 407 through 447, F. G. A. Stone and R. West editors, published by the Academic Press (New York, 1979). Persons skilled in the art can easily determine an effective amount of noble metal or platinum catalyst. Generally, an effective amount ranges from about 0.1 to 50 parts per million of the total organopolysiloxane composition.

The gels of the present invention are prepared either in a hydrosilylation compatible medium or solvent or an epoxy-gel formation compatible medium or solvent depending on the chemical nature of the gel being prepared. Both classes of preparation media include silicone solvents, preferably a silicone selected from the group consisting of cyclic silicones having the formula

D_{f}

where the subscript f is an integer ranging from about three to about 6 with D defined as

R⁴R⁵SiO_{2/2}

where R⁴ and R⁵ are each independently one to forty carbon atom monovalent hydrocarbon radicals and

linear silicones having the formula

M′D′ᵢM′

where D′ is defined as

R⁴R⁵SiO_{2/2}

where R⁴ and R⁵ are each independently one to forty carbon atom monovalent hydrocarbon radicals and M′ has the formula

R¹²R¹³R¹⁴SiO_{1/2}

where R¹², R¹³ and R¹⁴ are each independently one to forty carbon atom monovalent hydrocarbon radicals.

The hydrosilylation compatible medium or solvent are selected from the group consisitng of silicones and substituted silicones including: silicone oils of the desired viscosity from D₄ to 10,000 cps oils; polyethersilicone copolymers where the polyethers vary from 200 to 3000 molecular weight and may consist of alkylene oxide chains based on one, two or more types of monomer units such as ethylene oxide, propylene oxide or butylene oxide and may be attached to the silicone with 1 to six carbon chain, or through an silicone oxygen bond; polyester silicone copolymers; alkyl, aromatic or alkylaromatic substituted siloxanes; alkoxy substituted siloxanes including: substituted methoxy, ethoxy, propoxy, octyloxy, dodecanoxy, cetyryloxy or isostearyloxy siloxanes or other organically substituted siloxanes or siloxanes containing multiple organic substituents that are compatible with hydrosilylation reactions; hydro carbon solvents including: tetradecane, isododecane, isohexadecane, mineral oil, hydrogenate polydecene, apricot oil; ester solvents including: isopropyl myristate, diisopropyl adipate, isodecyl neopentanoate; ethers including: PPG-14 butyl ether, PPG 3 myristyl ether, ethoxylated alkylphenols; glyceryl esters of fatty acids including: sunflower oils, caprylic/capric triglyceride, C₁₀₋18 triglyceride; fatty acid glycerides including: glyceryl stearate, glyceryl dioleate; non-volatile fluorinated oil including: fluorinated silicones and fluorinated esters; aromatic solvents including; benzene, toluene and alkylbenzenes; and alcohols including: isopropanol, octanol, dodecanol, hexadecanol, cetearyl alcohol, isostearyl alcohol, myristyl alcohol.

The epoxy gel formation compatible medium or solvent is primarily defined by solvent inertness and is preferably selected from the group of silicone solvents D_{f} and M′D′ᵢM′ as previously defined and hydrocarbon solvents selected from the group consisting of paraffinic, iso-paraffinic, aromatic and alkyl aromatic solvents.

The compositions according to the present invention therefore advantageously comprise a stable dispersion of particles of at least one silicone in a dispersant medium preferably, a lipophilic phase, the lipophilic phase preferably selected from the group consisting of 1) physiologically acceptable liquid lipophilic or fatty phases and 2) silicone fluids selected from the group consisting of D_{f} and M′D′ᵢM′ where D_{f} and M′D′ᵢM′ are as previously defined.

These dispersions may in particular be provided in the form of nanoparticles of silicone gel in a stable dispersion in the said lipophilic or fatty phase. The nanoparticles are preferably of between 5 and 600 nm in size, given that above about 600 nm the dispersions of particles become much less stable. This size range includes all specific values and subranges therebetween, including 10, 25, 50, 100, 200, 300, 400 and 500 nm.

The liquid lipophilic or fatty phase in which the siloxane or silicone polymer may be dispersed may consist of any cosmetically or dermatologically acceptable, and more generally physiologically acceptable, oil chosen in particular from oils of inorganic, animal, plant or synthetic origin, carbonaceous oils, hydrocarbon oils, fluorinated oils and/or silicone oils, alone or in the form of a mixture insofar as they form a homogeneous and stable mixture and are compatible with the use envisaged. “Liquid fatty phase” refers to any nonaqueous medium which is liquid at room temperature. “Volatile fatty phase” refers to any nonaqueous medium capable of evaporating from the skin or the lips, at room temperature, in less than one hour.

Nonvolatile liquid lipophilic or fatty phase which can be used in the invention, include hydrocarbon oils such as paraffin oil or liquid petroleum jelly, vison oil, turtle oil, soya bean oil, perhydrosqualene, sweet almond oil, calophyllum oil, palm oil, grapeseed oil, sesame oil, maize oil, parleam oil, arara oil, rapeseed oil, sunflower oil, cottonseed oil, apricot oil, castor oil, avocado oil, jojoba oil, olive oil or cereal germ oil; esters of lanolic acid, oleic acid, lauric acid or stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl or linolenyl alcohol, isostearyl alcohol or octyl dodecanol; silicone oils such as polydimethylsiloxane (PDMS), which are optionally phenylated, such as phenyl trimethicones, or which are optionally substituted with optionally fluorinated aliphatic and/or aromatic groups, or with functional groups such as hydroxyl, thiol and/or amine groups; polysiloxanes modified with fatty acids, fatty alcohols or polyoxyalkylenes, fluorinated silicones and perfluorinated oils.

One or more oils which are volatile at room temperature and atmospheric pressure may optionally be used. These volatile oils have for example a steam pressure at ambient temperature and pressure of, preferably, from 1×10⁻³ to 300 mm Hg, provided that the boiling point is greater than 25° C. These volatile oils facilitate in particular the application of the composition to the skin, the mucous membranes and the superficial body growths. These oils may be hydrocarbon oils, silicone oils optionally comprising alkyl or alkoxy groups at the end of the silicone or pendant chain.

The volatile silicone oil which may be used in the invention, is selected from the group consisting of D_{f} and M′D′ᵢM′ as previously defined. The volatile oils represent preferably from 0 to 97.5% of the total weight of the composition, and more preferably from 5 to 85%. These ranges include all specific values and subranges therebetween, including 0.5, 1, 2, 8, 10, 15, 25, 30, 50, 60, 70, 80, 90 and 95% by weight.

Among the liquid lipophilic or fatty phases suitable for the compositions of the present invention are vegetable oils formed by esters of fatty acids and polyols, in particular triglycerides, such as sunflower, sesame or rapeseed oil, or the esters derived from long-chain acids or alcohols (that is to say having from 6 to 20 carbon atoms), in particular the esters of formula RCOOR′ in which R represents the residue of a higher fatty acid containing from 7 to 19 carbon atoms and R′ represents a hydrocarbon chain containing from 3 to 20 carbon atoms, such as palmitates, adipates and benzoates, in particular diisopropyl adipate. There may also be mentioned the hydrocarbons and in particular paraffin oils, liquid petroleum jelly, or hydrogenated polyisobutylene, isododecane, or alternatively the “ISOPARs”, volatile isoparaffins. There may also be mentioned the silicone oils such as polydimethylsiloxanes and polymethylphenylsiloxanes, optionally substituted with optionally fluorinated aliphatic and/or aromatic groups, or with functional groups such as hydroxyl, thiol and/or amine groups, and the volatile, in particular cyclic, silicone oils. There may also be mentioned the solvents, alone or in the form of a mixture, chosen from (i) linear, branched or cyclic esters having more than 6 carbon atoms, (ii) ethers having more than 6 carbon atoms, (iii) ketones having more than 6 carbon atoms. Monoalcohols having an overall solubility parameter according to the HANSEN solubility space of less than or equal to 20 (MPa)^{1/2} are understood to mean the aliphatic fatty alcohols having at least 6 carbon atoms, the hydrocarbon chain containing no substitution group. As mono-alcohols according to the invention, there may be mentioned oleyl alcohol, decanol, dodecanol, octadecanol and linoleyl alcohol.

Preferably the dispersant is selected from the group consisting of hydrocarbon oils, paraffin oil, liquid petroleum jelly, vison oil, turtle oil, soya bean oil, perhydrosqualene, sweet almond oil, calophyllum oil, palm oil, grapeseed oil, sesame oil, maize oil, parleam oil, arara oil, rapeseed oil, sunflower oil, cottonseed oil, apricot oil, castor oil, avocado oil, jojoba oil, olive oil, cereal germ oil; esters of lanolic acid, esters of oleic acid, esters of lauric acid, esters of stearic acid; isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate, diglyceryl triisostearate, myristic acid, palmitic acid, stearic acid, behenic acid, oleic acid, linoleic acid, linolenic acid, isostearic acid; cetanol, stearyl alcohol, oleyl alcohol, linoleyl or linolenyl alcohol, isostearyl alcohol or octyl dodecanol; silicone oils, polydimethylsiloxane, phenylated polydimethylsiloxane, polymethylphenylsiloxanes, phenyl trimethicones, phenyl trimethicones substituted with fluorinated aliphatic and/or aromatic groups, phenyl trimethicones substituted with functional groups such as hydroxyl, thiol and/or amine groups; polysiloxanes modified with fatty acids, fatty alcohols or polyoxyalkylenes; fluorinated silicones, perfluorinated oils, vegetable oils, sunflower oil, sesame oil, rapeseed oil, the esters long-chain acids or alcohols having the formula RCOOR′ in which R represents the residue of a higher fatty acid containing from 7 to 19 carbon atoms and R′ represents a hydrocarbon chain containing from 3 to 20 carbon atoms, hydrogenated polyisobutylene, isododecane, volatile isoparaffins, oleyl alcohol, decanol, dodecanol, octadecanol and linoleyl alcohol.

The choice of the non-aqueous medium is made by persons skilled in the art as a function of the nature of the monomers constituting the polymer and/or of the nature of the stabilizer, as indicated below. In particular, it is possible to use a polar or weakly polar oils such as vegetable oils of the long carbon chain-containing triglyceride type (apricot oil, jojoba oil) or the long carbon chain-containing esters such as octyldodecyl neopentanoate, the alkanes such as parleam oil, and the silicone oils. Furthermore, the total liquid lipophilic or fatty phase in which the polymer is dispersed may represent from 30% to 98% of the total weight of the composition and preferably from 30 to 75%. These ranges for the total liquid lipophilic or fatty phase include all specific values and sub-ranges therebetween, including 35, 40, 45, 50, 60, 70, 80, 85, 90 and 95% of the total weight of the composition. The nonvolatile part represents at least 0.5% and in practice from 1 to 30% of the total weight of the composition. These ranges for the nonvolatile part include all specific values and subranges therebetween, including 2, 3, 5, 10, 15, 20 and 25% of the total weight of the composition.

The personal care applications where the compositions of the present invention may be employed include, but are not limited to, deodorants, antiperspirants, antiperspirant/deodorants, shaving products, skin lotions, moisturizers, toners, bath products, cleansing products, hair care products such as shampoos, conditioners, mousses, styling gels, hair sprays, hair dyes, hair color products, hair bleaches, waving products, hair straighteners, manicure products such as nail polish, nail polish remover, nails creams and lotions, cuticle softeners, protective creams such as sunscreen, insect repellent and anti-aging products, color cosmetics such as lipsticks, foundations, face powders, eye liners, eye shadows, blushes, makeup, mascaras and other personal care formulations where silicone components have been conventionally added, as well as drug delivery systems for topical application of medicinal compositions that are to be applied to the skin.

In a preferred embodiment, the personal care composition of the present invention further comprises one or more personal care ingredients. Suitable personal care ingredients include, for example, emollients, moisturizers, humectants, pigments, including pearlescent pigments such as, for example, bismuth oxychloride and titanium dioxide coated mica, colorants, fragrances, biocides, preservatives, antioxidants, anti-microbial agents, anti-fungal agents, antiperspirant agents, exfoliants, hormones, enzymes, medicinal compounds, vitamins, salts, electrolytes, alcohols, polyols, absorbing agents for ultraviolet radiation, botanical extracts, surfactants, silicone oils, organic oils, waxes, film formers, thickening agents such as, for example, fumed silica or hydrated silica, particulate fillers, such as for example, talc, kaolin, starch, modified starch, mica, nylon, polymethylsilsequioxane, clays, such as, for example, bentonite and organo-modified clays.

Suitable personal care compositions are made by combining, in a manner known in the art, such as, for example, by mixing, one or more of the above components with the polyether siloxane copolymer network, preferably in the form of the silicone composition of the present invention. Suitable personal care compositions may be in the form of a single phase or in the form of an emulsion, including but not limited to oil-in-water, water-in-oil and anhydrous emulsions where the silicone phase may be either the discontinuous phase or the continuous phase, as well as multiple emulsions, such as, for example, oil-in water-in-oil emulsions and water-in-oil-in water-emulsions.

The compositions of the present invention may be utilized as prepared or as one or more components in emulsions. As is generally known, emulsions comprise at least two immiscible phases one of which is continuous and the other which is discontinuous. Further emulsions may be liquids with varying viscosities comprising solids. Additionally the particle size of the emulsions may render them microemulsions and when sufficiently small such microemulsions may be transparent. Further it is also possible to prepare emulsions of emulsions and these are generally known as multiple emulsions.

These primary types of emulsions may be:

1) aqueous emulsions where the discontinuous phase comprises water and the continuous phase comprises the polyether siloxane copolymer network of the present invention;

2) aqueous emulsions where the continuous phase comprises the polyether siloxane copolymer network of the present invention and the discontinuous phase comprises water;

3) non-aqueous emulsions where the discontinuous phase comprises a non-aqueous hydroxylic solvent and the continuous phase comprises the polyether siloxane copolymer network of the present invention; and

4) non-aqueous emulsions where the continuous phase comprises a non-aqueous hydroxylic organic solvent and the discontinuous phase comprises the polyether siloxane copolymer network of the present invention.

Non-aqueous emulsions comprising a silicone phase are described in U.S. Pat. Nos. 6,060,546 and 6,271,295 the disclosures of which are herewith and hereby specifically incorporated by reference.

As used herein the term “non-aqueous hydroxylic organic compound” means hydroxyl containing organic compounds as exemplified by but not limited to alcohols, glycols, polyhydric alcohols and polymeric glycols and mixtures thereof that are liquid at room temperature, e.g. about 25° C., and about one atmosphere pressure. The non-aqueous organic hydroxylic solvents are selected from the group consisting of hydroxyl containing organic compounds comprising alcohols, glycols, polyhydric alcohols and polymeric glycols and mixtures thereof that are liquid at room temperature, e.g. about 25° C., and about one atmosphere pressure. Preferably the non-aqueous hydroxylic organic solvent is selected from the group consisting of ethylene glycol, ethanol, propyl alcohol, iso-propyl alcohol, propylene glycol, dipropylene glycol, tripropylene glycol, butylene glycol, iso-butylene glycol, methyl propane diol, glycerin, sorbitol, polyethylene glycol, polypropylene glycol mono alkyl ethers, polyoxyalkylene copolymers and mixtures thereof.

Once the desired form is attained whether as a silicone only phase, an anhydrous mixture comprising the silicone phase (that may or may not contain so-called non-intended water), a hydrous mixture comprising the silicone phase, a water-in-oil emulsion, an oil-in-water emulsion, or either of the two non-aqueous emulsions or variations thereon, the resulting material is usually a high viscosity cream with good feel characteristics, and high absorbance of volatile solvent. It is capable of being blended into formulations for hair care, skin care, antiperspirants, sunscreens, cosmetics, color cosmetics, insect repellants, vitamin and hormone carriers, fragrance carriers and the like.

Colored materials suitable for use in the compositions of the present invention vary according to the laws of the country where the compositions are being sold because they involve topical application to human beings. However, even though categorized by the suitability for use in the United States (U.S.) or in the European Union (EU) the term colored materials includes all the colored materials in the following lists, lists A through D inclusive and all possible sub-combinations thereof:

List A. Certified organic colors listed for cosmetic uses in the U.S. and EU:

FD&C blue no. 1, FD&C green no. 3. FD&C red no. 4, FD&C red no. 40, FD&C yellow no. 5, F &C yellow no. 6, D&C blue no. 4. D&C brown no. 1, D&C green no. 5, D&C green no. 6, D&C green no. 8. D&C orange no. 4, D&C orange no. 5, D&C orange no. 10, D&C orange no. 11, D&C red no. 6, D&C red no. 7, D&C red no. 17, D&C red no. 21, D&C red no. 22, D&C red no. 27, D&C red no. 28, D&C red no. 30, D&C red no. 31, D&C red no. 33, D&C red no. 34, D&C red no. 36, D&C violet no. 2, D&C yellow no. 7, D&C yellow no. 8, D&C yellow no 10, D&C yellow no. 11, Ext. D&C violet no. 2, and Ext. D&C yellow no. 7.

List B. Inorganic colors listed for cosmetic uses in the U.S. and EU:

Iron oxide (red, yellow, black), Titanium dioxide, Zinc oxide, Ultramarine, Bismuth oxychloride, Chromium oxide green, Chromium hydroxide green, Ferric ferrocyanide, Manganese violet, and Guanine

List C. Additional only EU-approved colors list for cosmetic uses:

Acid green no. 1, Pigment yellow no.1, Pigment yellow no. 3, Solvent red no. 3, Solvent red no. 1, Pigment red no. 112, Pigment red no. 5, Acid orange no. 6, Acid red no. 14, Pigment red no. 68, Pigment red no.48, Acid red no. 27 & Al lake, Acid red no.18, Acid black no. 1, Pigment yellow no. 13, Solvent yellow no. 29, Acid red no. 73, Brilliant black no. 1, Acid blue no. 1, Acid blue no. 3, Basic violet no. 14, Basic blue no.26, Acid green no. 50, Acid red no. 52, Acid violet no. 9, Acid red no. 51, Pigment violet no.23, Pigment red no. 83. Acid blue no. 62, Acid blue no. 74, Pigment violet no. 19, Pigment blue no. 15, Direct blue no. 86, Pigment green no. 7, Bentonite, Barium sulfate, Calcium sulfate, Carbon black, Iron oxide (orange), Magnesium carbonate, Lactoflavin, Capsanthin, capsorubin, Beetroot red, Anthocyanins, Aluminum stearate, Zinc stearate, Magnesium stearate, Calcium stearate, Bromothymol blue, Bromocresol green, Acid red, Color Index (CI) 195, CI 18736, CI 18820, CI 18965, CI 20040, CI 21108, CI 24790, CI 27755, CI 40215, CI 40820, CI 40825, CI 40850, CI 42080, CI 42090, CI 42100, CI 42170, CI 42520, CI 42735, CI 45220, CI 45396, CI 45405, CI 50325, CI 50420, CI 60724, CI 61585, CI 69800, CI 69825, CI 71105, CI 73000, CI 73385, CI 73915, CI 74100, CI 75100, CI 75125, CI 75135, CI 75300, CI 77002, CI 77015, CI 77220, CI 77267, CI 77268:1, CI 77346, CI 77480, and CI 77745

List D. Other colors list for cosmetic uses:

Beta carotene, Annatto, Caramel, Carmine. Chlorophyllin—copper complex, Henna, Aluminum powder, Bronze or copper powder, Silver, Mica, and Titanated mica.

Experimental

Panel 1

Lipstick compositions comprising the ingredients listed in Table I were prepared by combining dimethicone copolyol (PEG/PPG-20/15 Dimethicone) in D₅ (decamethyl-cyclo-penta-siloxane), isododecane and the indicated silicone gel under high shear mixing at ambient temperatures in a first container. In a second container, C₁₈₋₃₆ acid triglyceride, Ozokerite®, polyethylene and pigments were combined and heated to 70° C. After the mixture in the second container was melted the silicone containing phase in the first container was slowly added to the organic phase(s) in the second container. Mixing was continued for an additional 30 minutes to ensure homogeneity.

TABLE I
Compositions for Panel 1 Lipsticks

| | | | | 4 |
|---|---|---|---|---|
| Formulation | 1 | 2 | 3 | (control) |
| Ingredients, Wt. % | | | | |
| 40 Wt. % dimethicone copolyol in D₅ | 10 | 10 | 10 | 10 |
| C₁₈₋₃₆ acid triglyceride | 5 | 5 | 5 | 5 |
| Ozokerite ® | 3 | 3 | 3 | 3 |
| Polyethylene | 5 | 5 | 5 | 5 |
| Isododecane | 20 | 20 | 20 | 20 |
| D & C Red #7 Ca Lake | 7 | 7 | 7 | 7 |
| Silicone Gel A | 50 | 0 | 0 | 0 |
| Silicone Gel B | 0 | 50 | 0 | 0 |
| Silicone Gel C | 0 | 0 | 50 | 0 |
| Silicone D | 0 | 0 | 0 | 50 |

Notes:
C₁₈₋₃₆ acid triglyceride is a triester of glycerin and C18-36 acid available from Croda, Inc. of 7 Century Drive, Parsippany, NJ 07054.
Ozokerite is a hydrocarbon wax derived from mineral or petroleum available from Strahl & Pitsch, Inc. of 230 Great East Neck Rd., West Babylon, NY 11704.
Polyethylene is a polymer of ethylene available from New Phase Technologies of 377 Hoes Lanes, Piscataway, NJ.
Silicone Gel A is an example of Silicone Gel I and is commercially available as SFE-839 from GE Silicones, 260 Hudson River Road, Waterford, NY 12188.
Silicone Gel B is an example of Silicone Gel VII, an experimental sample.
Silicone Gel C is an example of Silicone Gel II, an experimental sample.
Silicone D is decamethyl-cyclo-penta-siloxane (D₅) and is commercially available as SF-1202 from GE Silicones, 260 Hudson River Road, Waterford, NY 12188.

The lipsticks were tested for transfer resistance using the following method. Seventy mg of the tested lipsticks was uniformly applied to a 2″×3″ pork intestine slide and air dried for 4 hours. A white T-shirt was placed on the surface of the lipstick coated pork intestine slide and pressed with a 500 g weight by rotating the weight in a 360 degree or one circular motion. The amount of lipstick transferring to the T-shirt was measured by a ColorEye 7000 (available from Gretag-Macbeth, 617 Little Britain Rd., New Windsor, N.Y. 12553). The “a” value on the Hunter L, a, b color scale was used as an indicator of rub-out and thus transfer resistance. The “L” value is the measurement on a black and white scale (0-100) where the higher the number the whiter the color, with zero representing black, and 100 representing white. The “a” value represents a red-green color scale where a positive number represents redness and a negative number represents greenness. The “b” value represents a blue-yellow color scale where a positive number represents yellowness and a negative number represents blueness. In these examples, the “a” values were used as an indicator of rub-out because the only colored material used was D&C red #7 Ca Lake. Since only a red coloring material was used, the “L” and “b” number does not reflect any transfer and was not used for any experimental evaluation. The average “L,” “a,” “b” values (average of 5 samples per formulations) are shown in Table II.

Transfer resistance as it applies to the silicone gel comprising formulations of the present invention is more quantitatively defined by a comparison to a control formulation that does not contain a silicone gel material where both the control and candidate formulation(s) have the same weight percent dye or pigment in the cosmetic formulation. Thus a percent reduction in the intensity of color transferred can be computed using the following formula (based on “a” values of the L, a, b color scale):

Percent transfer reduction=100(a_{control}−a_{tested formulation}/a_{control})

where a_{control} is the “a” value on L,a,b scale of control formulation,

a_{tested formulation} is the a value on L,a,b scale of color cosmetic containing silicone gel. The same formula can be used for the yellow (positve b)-blue (negative b) color couple, “b” values, and green color (negative values of “a”) making the proper algebraic adjustments when the scaled numbers are negative numbers.

TABLE II
Transfer Measurements for Experimental Lipsticks

| | | | | Percent |
|---|---|---|---|---|
| | | | | Transfer |
| Sample | L | a | b | Reduction |
| 1 | 91.52 | 7.02 | −11.36 | 43% |
| 2 | 91.16 | 7.43 | −11.22 | 40% |
| 3 | 90.15 | 8.18 | −11.34 | 34% |
| 4 (control) | 83.18 | 12.36 | −10.36 | — |

Lipstick formulations containing silicone gels show the lower numbers for the “a” value indicating a lower level of color transferred to the T-shirt during the rub-out testing, i.e. a higher transfer resistance, i.e. a greater percent transfer reduction. Qualitative evaluations were also made of the transfer resistance of the lipstick formulations. Samples 1 and 2 gave slightly better transfer resistance than sample 3. By comparison to the control, all the silicone gel containing lipstick formulations provided excellent rub-out protection. These results are consistent with the quantitative values for “a.” After three days at room temperature, syneresis was noticeable in the control formulation but in contrast the silicone gel containing samples did not exhibit any syneresis.

Panel 2

A lipstick comprised of the ingredients listed below was prepared by combining 40% dimethicone copolyol in D5, isododecane, D5, and the designated silicone gel under high shear mixing at ambient temperature. In a separate container, C₁₈₋₃₆ acid triglyceride, Ozokerite, polyethylene and pigments were combined and heated to 70° C. After the mixture was melted, the silicone phase was added slowly to the organic phase. The mixing was continued for an additional 30 minutes to ensure homogeneity.

TABLE 3
Compositions of Panel 2 Lipsticks

| | Wt % | Wt % | Wt % | Wt % |
|---|---|---|---|---|
| Ingredients | 5 (Control) | 6 | 7 | 8 |
| 40% Dimethicone copolyol in | 10 | 10 | 10 | 10 |
| D₅ | | | | |
| C₁₈₋₃₆ acid triglyceride | 6 | 6 | 6 | 6 |
| Ozokerite | 4 | 4 | 4 | 4 |
| Polyethylene | 5 | 5 | 5 | 5 |
| Isododecane | 5 | 5 | 5 | 5 |
| 25% D & C Red #7 Ca lake in | 28 | 28 | 28 | 28 |
| castor oil | | | | |
| Silicone D (SF1202) | 42 | 22 | 22 | 22 |
| Silicone Gel E | | 20 | | |
| Silicone Gel F (DC9040) | | | 20 | |
| Silicone Gel A | | | | 20 |

Notes:
Silicone Gel E is an example of Silicone Gel II, an experimental sample.
Silicone Gel F (DC9040) is an example of Silicone Gel VI and is commercially available the Dow-Corning Corporation in Midland, MI.

The lipsticks were tested for transfer resistance using the method previously described. Seventy mg of the tested lipsticks was uniformly applied to the 2″×3″ pork intestine slides and aired dried for 4 hours. A white T-shirt was placed on the surface of the lipstick coated pork intestine slide and pressed with 500 g weight by rotating the weight in a 360 degree or one circular motion. The amount of lipstick transferring to the t-shirt was measured by a ColorEye 7000 (available from Gretag-Macbeth, 617 Little Britain Rd., New Windsor, N.Y. 12553.) The “a” value on the Hunter L, a, b color scale was used as an indicator of rub-out and thus transfer resistance. The “L” value is the measurement on a black and white scale (0-100) where the higher the number the whiter the color, with 0 representing black and 100 representing white. The a value represents red and green color where the positive number reflects the red color and the negative number represents green color. In this study the lower a value represents less transfer and is more desirable. The b value represents blue and yellow and the positive number represent yellow and negative number represents blue. In this study the a value was used as an indicator of rub out magnitude due to the presence of D&C red #7 Ca lake in the formulations. Since only red coloring was used the L and b number does not reflect transfer in this particular instance and was not used for evaluating the formulations. The average Lab value results were shown in the table below.

TABLE 4
Transfer Resistance Values for Panel 2 Lipsticks

| | | | | | |
|---|---|---|---|---|---|
| | | | | Percent | |
| Samples | L | a | b | Reduction | Transfer |

| | | | | |
|---|---|---|---|---|
| 5 (Control) | 83.86 | 13.12 | −9.62 | — |
| 6 | 84.08 | 10.86 | −9.89 | 17% |
| 7 | 81.33 | 12.19 | −9.54 | 7% |
| 8 | 82.23 | 10.41 | −10.32 | 20% |

In this examples the primary indicator of transfer resistance is the “a” value. Lipsticks containing silicone gel show lower numbers of a value, which demonstrates the lower color transfer of the lipsticks from the tested slide to T-shirt during the rub-out testing. The ranking of transfer resistance from high to low is as follows: Sample 8> Sample 6> Sample 7.

Although these tests measured lipstick formulations other color cosmetic such as lipsticks, foundations, face powders, eye liners, eye shadows, blushes, makeup, and mascaras although not limited thereto may be similarly measured by a red-green or blue-yellow color scale as herein defined. As measured by the previously defined test, the transfer resistant color cosmetic formulations of the present invention exhibit a percent transfer reduction value of 7% or greater, preferably 17% or greater, more preferably 20% or greater, and most preferably 34% or greater.

Cosmetic formularies list many different color cosmetic formulations where waxes or other solid materials and cosmetic fluids also including silicone oils may be replaced by the silicone gels of the present invention gels I through VII), thus imparting transfer resistance to the formulation, e.g.

Liquid Foundation

This light weight skin feel liquid foundation may be prepared by mixing part A together and heat to 65° C. Part B is separately mixed and heated to 60 degree C. Then part A is slowly added to part B.

| | wt % |
|---|---|
| Part A | |
| Water | q.s. |
| Laureth-9 | 1.0 |
| Butylene glycol | 3.0 |
| Magnesium aluminum silicate | 0.25 |
| Potassium cetyl phosphate | 1.5 |
| Preservatives | 0.5 |
| Part B | |
| 40% PEG/PPG-20/15 dimethicone | 7.5 |
| in cyclopentasiloxane | |
| Titanium dioxide | 8.75 |
| Red iron oxide | 0.5 |
| Yellow iron oxide | 1.0 |
| Black iron oxide | 0.07 |
| Talc | 5.0 |
| Caprylic/capric triglyceride | 6.0 |
| Cetyl alcohol | 1.25 |
| C30-45 alkyl dimethicone | 2.0 |
| Cyclopentasiloxane | 12.0 |
| Sorbitan laurate | 2.5 |
| Silicone Gel of the present invention | 7.5 |

Powder Blusher

This powder blusher provides a soft silky feel with good adhesion. It is (may be) prepared by mixing all ingredients of part A together under a high speed mixer. Then part B is added to part A.

| Composition | wt % |
|---|---|
| Part A | |
| Talc | q.s. |
| Sericite | 10.0 |
| Bismuth oxychloride | 6.0 |
| Polymethyl silsesquioxane (Tospearl 145A) | 10.0 |
| Magnesium myristate | 3.0 |
| Calcium silicate | 0.2 |
| D & C Red No. 30 Lake | 1.0 |
| Yellow iron oxide | 0.5 |
| Red iron oxide | 0.25 |
| Methylparaben | 0.2 |
| Propylparaben | 0.1 |
| Imidazolidinyl urea | 0.25 |
| Part B | |
| Silicone Gel of the present invention | 5.0 |
| Bis-phenylpropyl dimethicone | 1.5 |
| Eyeshadow/Eyeliner pencil | |

Eyeshadow/eyeliner pencil is made by melting waxes, oils, and Silicone Gel of the present invention at 75 C. Pigment is added to the melted waxes. The formulation is passed through three-roll mill to disperse pigment. It is then extruded through a single orifice to form pencil lead.

| Composition | wt % |
|---|---|
| Japan wax | 30.0 |
| Ceresin | 15.0 |
| Microcrystalline wax | 4.0 |
| Cetearyl methicone | 6.0 |
| Glyceryl triisostearate | 3.0 |
| Jojoba oil | 15.0 |
| Sunflower seed oil | 5.0 |
| Silicone Gel of the present invention | 5.0 |
| Pigment | 17.0 |
| Mascara | |

This oil in water emulsion mascara is prepared by heating Part A to 75 C. under high speed homogenizer. Part B is combined and heated to 85 C. Emulsion is developed by adding part B to part A while homogenizing.

| Composition | wt % |
|---|---|
| Part A | |
| Deionized water | q.s. |
| PVP | 2.0 |
| Hydroxyethylcellulose | 1.0 |
| Triethanolamine | 2.0 |
| Methylparaben | 0.3 |
| Disodium EDTA | 0.1 |
| Black iron oxide | 10.0 |
| Part B | |
| Stearic acid | 4.5 |
| Glyceryl stearate | 2.0 |
| Silicone Gel of the present invention | 7.0 |
| C₃₀₋₄₅ alkyl dimethicone | 4.5 |
| Propylparaben | 0.1 |
| Acrylate copolymer | 20.0 |
| DMDM Hydantoin | 0.18 |
| Anhydrous blush | |

This silky blush is prepared by combining color grind, waxes, oils, and Silicone Gel of the present invention, and heating to 80° C. Talc, polymethylsilsesquioxane and mica are added to the batch and mix until uniform.

| Composition | wt % |
|---|---|
| 50% D & C Red No. 6 Barium lake | 2.0 |
| in Bis-phenyl propyl dimethicone | |
| C18-36 acid triglyceride | 12.1 |
| Glyceryl tribehenate | 1.9 |
| Phenyl trimethicone | 30.2 |
| Caprylic/capric triglyceride | 3.0 |
| Polyglyceryl-3 diisostearate | 0.5 |
| Silicone gel | 5.0 |
| Methylparaben | 0.2 |
| Propylparaben | 0.1 |
| Talc | 18.0 |
| Polymethylsilsesquioxane | 15.0 |
| Mica | 12.0 |
| Pressed Powder | |

The facial pressed powder is made by blending and milling all the powders and colors together. When the desired uniformity and particle size of the batch is achieved, a blend of dimethicone (5 cSt) and Silicone Gel of the present invention is sprayed onto the batch. It is then pressed in suitable containers.

| Composition | wt % |
|---|---|
| Talc | 36.4 |
| Boron nitride | 30.0 |
| Titanium dioxide | 15.0 |
| Yellow iron oxide | 2.8 |
| Red iron oxide | 1.3 |
| Black iron oxide | 1.2 |
| Zinc stearate | 3.0 |
| Methylparaben | 0.2 |
| Propylparaben | 0.1 |
| Dimethicone 5 cSt | 4.0 |
| Silicone Gel of the present invention | 6.0 |

Having described the invention that which is claimed is:

1. A colored cosmetic composition comprising:
(a) a silicone gel;
(b) a dispersant medium; and
(c) a colored material
whereby said colored cosmetic composition is transfer resistant.

2. The colored cosmetic composition of claim 1 wherein the silicone gel is selected from the group of gels consisting of:
(i) a gel formed from a silicone and a hydrosilylation compatible solvent wherein said silicone is prepared by the hydrosilylation of a linear alkenyl polyorganosiloxane and a hydride resin;
(ii) a gel formed as a reaction product of an epoxy functional hydrido-siloxane said reaction product being formed in an epoxy-gel formation compatible solvent;
(iii) a gel formed from a silicone and a hydrosilylation compatible solvent wherein said silicone is prepared by the hydrosilylation of a linear hydrogen polyorganosiloxane and an alkenyl resin;
(iv) a gel formed from a silicone and a hydrosilylation compatible solvent wherein said silicone is prepared by the hydrosilylation of a linear hydrogen polyorganosiloxane and a linear alkenyl polyorganosiloxane;
(v) a gel formed from a silicone and hydrosilylation compatible solvent wherein said silicone is prepared by the hydrosilylation of a hydrogen polyorganosiloxane resin and an alkenyl polyorganosiloxane resin;
(vi) a gel formed from a silicone and a hydrosilylation compatible solvent wherein said silicone is prepared by the hydrosilylation of a linear hydrogen organopoly-siloxane having two or more hydride functionalities per molecule and an α, ω reactive organic molecule possessing two or more reactive functionalities per molecule; and
(vii) a gel formed as a reaction product of a vinyl functional hydrido-siloxane in a hydrosilylation compatible solvent.

3. The colored cosmetic composition of claim 1 wherein the dispersant medium is selected from the group consisting of physiologically acceptable liquid lipophilic or fatty phases and silicone fluids.

4. The colored cosmetic composition of claim 3 wherein the silicone gel is selected from the group of gels consisting of:
(i) a gel formed from a silicone and a hydrosilylation compatible solvent wherein said silicone is prepared by the hydrosilylation of a linear alkenyl polyorganosiloxane and a hydride resin;
(ii) a gel formed as a reaction product of an epoxy functional hydrido-siloxane said reaction product being formed in an epoxy-gel formation compatible solvent;
(iii) a gel formed from a silicone and a hydrosilylation compatible solvent wherein said silicone is prepared by the hydrosilylation of a linear hydrogen polyorganosiloxane and an alkenyl resin;
(iv) a gel formed from a silicone and a hydrosilylation compatible solvent wherein said silicone is prepared by the hydrosilylation of a linear hydrogen polyorganosiloxane and a linear alkenyl polyorganosiloxane;
(v) a gel formed from a silicone and hydrosilylation compatible solvent wherein said silicone is prepared by the hydrosilylation of a hydrogen polyorganosiloxane resin and an alkenyl polyorganosiloxane resin;
(vi) a gel formed from a silicone and a hydrosilylation compatible solvent wherein said silicone is prepared by the hydrosilylation of a linear hydrogen organopolysiloxane having two or more hydride functionalities per molecule and an α, ω reactive organic molecule possessing two or more reactive functionalities per molecule; and
(vii) a gel formed as a reaction product of a vinyl functional hydrido-siloxane in a hydrosilylation compatible solvent.

5. The colored cosmetic composition of claim 1 wherein the colored cosmetic is selected from the group consisting of lipsticks, foundations, face powders, eye liners, eye shadows, blushes, makeup, and mascara.

6. The colored cosmetic composition of claim 2 wherein the colored cosmetic is selected from the group consisting of lipsticks, foundations, face powders, eye liners, eye shadows, blushes, makeup, and mascara.

7. The colored cosmetic composition of claim 3 wherein the colored cosmetic is selected from the group consisting of lipsticks, foundations face powders, eye liners, eye shadows, blushes, makeup, and mascara.

8. The colored cosmetic composition of claim 4 wherein the colored cosmetic is selected from the group consisting of lipsticks, foundations, face powders, eye liners, eye shadows, blushes, makeup, and mascara.

9. A colored cosmetic composition comprising:
a silicone gel selected from the group of silicone gels consisting of:
(i) a gel formed from a silicone and a hydrosilylation compatible solvent wherein said silicone is prepared by the hydrosilylation of a linear alkenyl polyorganosiloxane and a hydride resin;
(ii) a gel formed as a reaction product of an epoxy functional hydrido-siloxane said reaction product being formed in an epoxy-gel formation compatible solvent;
(iii) a gel formed from a silicone and a hydrosilylation compatible solvent wherein said silicone is prepared by the hydrosilylation of a linear hydrogen polyorganosiloxane and an alkenyl resin;
(iv) a gel formed from a silicone and a hydrosilylation compatible solvent wherein said silicone is prepared by the hydrosilylation of a linear hydrogen polyorganosiloxane and a linear alkenyl polyorganosiloxane;
(v) a gel formed from a silicone and hydrosilylation compatible solvent wherein said silicone is prepared by the hydrosilylation of a hydrogen polyorganosiloxane resin and an alkenyl polyorganosiloxane resin;
(vi) a gel formed from a silicone and a hydrosilylation compatible solvent wherein said silicone is prepared by the hydrosilylation of a linear hydrogen organopolysiloxane having two or more hydride functionalities per molecule and an α, ω reactive organic molecule possessing two or more reactive functionalities per molecule; and
(vii) a gel formed as a reaction product of a vinyl functional hydrido-siloxane in a hydrosilylation compatible solvent;
(b) a dispersant medium selected from the group consisting of hydrocarbon oils, paraffin oil, liquid petroleum jelly, vison oil, turtle oil, soya bean oil, perhydrosqualene, sweet almond oil, calophyllum oil, palm oil, grapeseed oil, sesame oil, maize oil, parleam oil, arara oil, rapeseed oil, sunflower oil, cottonseed oil, apricot oil, castor oil, avocado oil, jojoba oil, olive oil, cereal germ oil; esters of lanolic acid, esters of oleic acid, esters of lauric acid, esters of stearic acid; isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate, diglyceryl triisostearate, myristic acid, palmitic acid, stearic acid, behenic acid, oleic acid, linoleic acid, linolenic acid, isostearic acid; cetanol, stearyl alcohol, oleyl alcohol, linoleyl or linolenyl alcohol, isostearyl alcohol or octyl dodecanol; silicone oils, polydimethylsiloxane, phenylated polydimethylsiloxane, polymethylphenylsiloxanes, phenyl trimethicones, phenyl trimethicones substituted with fluorinated aliphatic and/or aromatic groups, phenyl trimethicones substituted with functional groups such as hydroxyl, thiol and/or amine groups; polysiloxanes modified with fatty acids, fatty alcohols or polyoxyalkylenes; fluorinated silicones, perfluorinated oils, vegetable oils, sunflower oil, sesame oil, rapeseed oil, the esters long-chain acids or alcohols having the formula RCOOR′ in which R represents the residue of a higher fatty acid containing from 7 to 19 carbon atoms and R′ represents a hydrocarbon chain containing from 3 to 20 carbon atoms, hydrogenated polyisobutylene, isododecane, volatile isoparaffins, oleyl alcohol, decanol, dodecanol, octadecanol and linoleyl alcohol.
(d) a colored material selected from the group consisting of FD&C blue no. 1, FD&C green no.3, FD&C red no. 4 ,FD&C red no. 40, FD&C yellow no. 5, FF&C yellow no. 6, D&C blue no. 4, D&C brown no. 1, D&C green no. 5, D&C green no. 6, D&C green no. 8, D&C orange no. 4, D&C orange no. 5, D&C orange no. 10, D&C orange no. 11, D&C red no. 6, D&C red no. 7, D&C red no. 17, D&C red no. 21, D&C red no. 22, D&C red no. 27, D&C red no. 28, D&C red no. 30, D&C red no. 31, D&C red no. 33, D&C red no. 34, D&C red no. 36, D&C violet no. 2, D&C yellow no. 7, D&C yellow no. 8, D&C yellow no. 10, D&C yellow no. 11, Ext. D&C violet no. 2, Ext. D&C yellow no. 7, Iron oxide (red, yellow, black), Titanium dioxide, Zinc oxide, Ultramarine, Bismuth oxychloride, Chromium oxide green, Chromium hydroxide green, Ferric ferrocyanide, Manganese violet, Guanine, Acid green no. 1, Pigment yellow no.1, Pigment yellow no. 3, Solvent red no. 3, Solvent red no. 1, Pigment red no. 112, Pigment red no. 5, Acid orange no. 6, Acid red no. 14, Pigment red no. 68, Pigment red no.48, Acid red no. 27 & Al lake, Acid red no.18, Acid black no. 1, Pigment yellow no. 13, Solvent yellow no. 29, Acid red no. 73, Brilliant black no. 1, Acid blue no. 1, Acid blue no. 3, Basic violet no. 14, Basic blue no.26, Acid green no. 50, Acid red no. 52, Acid violet no. 9, Acid red no. 51, Pigment violet no.23, Pigment red no. 83, Acid blue no. 62, Acid blue no. 74, Pigment violet no. 19, Pigment blue no. 15, Direct blue no. 86, Pigment green no. 7, Bentonite, Barium sulfate, Calcium sulfate, Carbon black, Iron oxide (orange), Magnesium carbonate, Lactoflavin, Capsanthin, capsorubin, Beetroot red, Anthocyanins, Aluminum stearate, Zinc stearate, Magnesium stearate, Calcium stearate, Bromothymol blue, Bromocresol green, Acid red, Color Index (CI) 195, CI 18736, CI 18820, CI 18965, CI 20040, CI 21108, CI 24790, CI 27755, CI 40215, CI 40820, CI 40825, CI 40850, CI 42080, CI 42090, CI 42100, CI 42170, CI 42520, CI 42735, CI 45220, CI 45396, CI 45405, CI 50325, CI 50420, CI 60724, CI 61585, CI 69800, CI 69825, CI 71105, CI 73000, CI 73385, CI 73915, CI 74100, CI 75100, CI 75125, CI 75135, CI 75300, CI 77002, CI 77015, CI 77220, CI 77267, CI 77268:1, CI 77346, CI 77480, CI 77745, Beta carotene, Annatto, Caramel, Carmine, Chlorophyllin—copper complex, Henna, Aluminum powder, Bronze or copper powder, Silver, Mica, and Titanated mica;
whereby said colored cosmetic composition is transfer resistant.

10. The colored cosmetic composition of claim 9 wherein the colored cosmetic is selected from the group consisting of lipsticks, foundations, face powders, eye liners, eye shadows, blushes, makeup, and mascara;

11. The colored cosmetic composition of claim 10 wherein the silicone gel is a gel formed from a silicone and a hydrosilylation compatible solvent wherein said silicone is prepared by the hydrosilylation of a linear alkenyl polyorganosiloxane and a hydride resin.

12. The colored cosmetic composition of claim 10 wherein the silicone gel is a gel formed as a reaction product of an epoxy functional hydrido-siloxane said reaction product being formed in an epoxy-gel formation compatible solvent.

13. The colored cosmetic composition of claim 10 wherein the silicone gel is a gel formed from a silicone and a hydrosilylation compatible solvent wherein said silicone is prepared by the hydrosilylation of a linear hydrogen polyorganosiloxane and an alkenyl resin.

14. The colored cosmetic composition of claim 10 wherein the silicone gel is a gel formed from a silicone and a hydrosilylation compatible solvent wherein said silicone is prepared by the hydrosilylation of a linear hydrogen polyorganosiloxane and a linear alkenyl polyorganosiloxane.

15. The colored cosmetic composition of claim 10 wherein the silicone gel is a gel formed from a silicone and hydrosilylation compatible solvent wherein said silicone is prepared by the hydrosilylation of a hydrogen polyorganosiloxane resin and an alkenyl polyorganosiloxane resin.

16. The colored cosmetic composition of claim 10 wherein the silicone gel is a gel formed from a silicone and a hydrosilylation compatible solvent wherein said silicone is prepared by the hydrosilylation of a linear hydrogen organopolysiloxane having two or more hydride functionalities per molecule and an α, ω reactive organic molecule possessing two or more reactive functionalities per molecule.

17. The colored cosmetic composition of claim 10 wherein the silicone gel is a gel formed as a reaction product of a vinyl functional hydrido-siloxane in a hydrosilylation compatible solvent.

18. A colored cosmetic composition consisting essentially of:
(a) a silicone gel;
(b) a dispersant medium; and
(c) a colored material
whereby said colored cosmetic composition is transfer resistant.

19. A colored cosmetic composition consisting of:
(a) a silicone gel;
(b) a dispersant medium; and
(c) a colored material
whereby said colored cosmetic composition is transfer resistant.
